(19) United States
(12) Patent Application Publication
(10) Pub. No.: US 2016/0310625 A1
(43) Pub. Date: Oct. 27, 2016

(54) DEVICE AND METHOD FOR SUSTAINED RELEASE OF FRAGRANT COMPOUND

(71) Applicant: NANOMEDICAL SYSTEMS, INC., Austin, TX (US)

(72) Inventors: Lee HUDSON, Elgin, TX (US); Randal GOODALL, Austin, TX (US); David SAATHOFF, Austin, TX (US)

(73) Assignee: NANOMEDICAL SYSTEMS, INC., Austin, TX (US)

(21) Appl. No.: 15/102,915

(22) PCT Filed: Dec. 9, 2014
(86) PCT No.: PCT/US2014/069221

§ 371 Date: Jun. 9, 2016

Related U.S. Application Data
(60) Provisional application No. 61/914,101, filed on Dec. 10, 2013.

(51) Int. Cl. A61L 9/12; A61L 9/14; H05K 5/00; H05K 5/03; A45D 34/02; A44C 15/00

(52) U.S. Cl. CPC A61L 9/12; A45D 34/02; A44C 15/002; H05K 5/0086; H05K 5/03; A61L 9/14; A45D 2034/007

(57) ABSTRACT

Devices and methods for long-term sustained release of a fragrant compound. Exemplary embodiments may comprise a channeled member with nanochannels or microchannels to provide a sustained release of the fragrant compound.

The present application claims the priority benefit of U.S. provisional application No. 61/914,101, filed Dec. 10, 2013, the entire contents of which are incorporated herein by reference.

### BACKGROUND INFORMATION

Fragrant compounds, including for example, perfumes and colognes, have long been used to provide a pleasant scent for a user. A fragrance can be used to scent a variety of spaces, including room, vehicle, and personal. For personal space, fragrant compounds are often conventionally applied at pulse points, e.g. behind the ears, the nape of the neck, the insides of wrists, elbows and knees. The pulse point can warm the perfume and somewhat extend the release of the fragrant compound over time. However, even with the increased fragrance release resulting from increased temperature, the scent level will significantly subside over time after the initial application.

Conventional fragrances, even those deemed high quality, are liquids comprising a majority of aromatic solvent (60-70% or more), primarily ethanol and water. Mixtures of fragrant substances with various volatilities make up the remainder of the fragrance. These are often grouped into broad release timings as “top notes”, “middle notes”, and “base notes” aromas. The sensation of a fragrance then is first ethanol, followed by a few minutes of the top notes, followed by tens to hundreds of minutes of the middle notes, with a residual of a few hours of the base notes. Therefore the perception of the fragrance both diminishes and degrades over time.

With conventional applications, fragrant compounds such as perfume, cologne or eau de toilette typically provide a noticeable scent for only a few hours. The decrease in the scent level can necessitate re-application of the fragrant compound in order to provide the desired fragrance level for an extended period of time. Another alternative is for the user to initially apply a level of fragrant compound that significantly exceeds the desired level and eventually reduces to the desired level over time. Such efforts are only somewhat effective in extending the effective time range of the fragrant compounds and include overwhelming initial scent, excessive consumption of the possibly expensive fragrance, and inconveniences to the user.

It is therefore desirable to provide an effective sustained release of fragrant compound without necessitating frequent re-application or initially high fragrance levels.

The issues raised above are merely exemplary of the potential shortcomings of existing systems and are not intended to be an exhaustive listing of the issues addressed by embodiments of the present disclosure.

### SUMMARY

Exemplary embodiments of the present disclosure can provide for a sustained release of a fragrant compound without requiring frequent re-applications or unpleasantly high initial aroma levels. Exemplary embodiments can also be conveniently coupled to personal articles such as clothing or jewelry. For example, the device can include a coupling mechanism (e.g. a clasp or ring) that allows the device to be coupled to the personal article. Exemplary embodiments can also be conveniently incorporated (e.g. inserted) into other personal articles in the vicinity of the user. For example, the device can be included within a fashion accessary (e.g., a belt buckle) or a personal article in the user's grasp (e.g., an electronic handheld device, including for example, a cell phone, tablet, or laptop computer or a protective or decorative covering thereof).

As explained in further detail below, exemplary embodiments can comprise a reservoir in fluid communication with a channeled member that is configured to provide a sustained release of a fragrant compound over a significant period of time. The channeled member may include nanochannels and/or microchannels that control the release rate of the fragrant compound and provides for a more consistent aromatic level in the environment surrounding the user. In certain embodiments, the channels can be dimensioned to control the release rate of molecules of the fragrant compound as the compound evaporates and the molecules diffuse into the surrounding atmosphere.

In exemplary embodiments, the release of the fragrant compound by a channeled member can occur in one or more modes. For example, in a first mode, the fragrant compound in the reservoir in fluid form can move under the influence of a force, for example, a gas pressure within the reservoir or by gravity acting on the fragrant compound fluid, through the channeled member, so that the fluid arrives at the external face of the channeled member from whence it may evaporate into the surrounding atmosphere. This mode can be realized for example under a gravitational force in embodiments in which the reservoir is spatially disposed above the channeled member, such that fluid surface inside the reservoir is directly coupled to the channeled member. In this mode, the evaporation from the surface of the channeled member may additionally drive a capillary force to continuously draw fluid from the reservoir through the channeled member.

In a second mode, as in the first mode, the fragrant compound may collect on the surface of the channeled member in such quantity that an additional evaporative diffuser may be disposed in direct contact with or proximate to the channeled member such that the fragrant compound released through the channeled member soaks into and moves through the evaporative diffuser and evaporates from its surface into the surrounding atmosphere. This mode can be realized for example in embodiments in which the reservoir is spatially disposed above the channeled member and the evaporative diffuser is disposed below the channeled member. The evaporative diffuser can be of any suitable material, including without limitation paper, fabric, fibrous material including polymeric fibrous material, hydrophilic or hydrophobic materials, and plural layers of such materials.

In a third mode, the fragrant compound in the reservoir in fluid form can evaporate, so that the gaseous form of the compound is released through the channeled member into the surrounding atmosphere. This mode can be realized for example in embodiments in which the reservoir is spatially located lower than the channeled member, such that fluid surface inside the reservoir is weakly coupled or intermittently coupled to the channeled member. Because the mass transport of the fragrant compound is primarily via the gas, ceteris paribus, this mode of operation is typically several times slower than the first and second described modes. This allows for a method of usage of the fragrance release device in which the device may be inverted from the first or second mode into the third mode as a simple means of “turning off” the device, whereby the release of fragrant compound is greatly reduced.

In certain embodiments, the device may also serve as a decorative or ornamental item, while in other embodiments, the device may be concealed so that it is not noticeable. In other embodiments, the device may be incorporated within a personal article.

In certain embodiments, the device may be filled by accessing a port into the reservoir which may comprise an attached or separate lid portion that seals to the remaining portion of the reservoir body to form a seal tight enough to prohibit the leakage of the fragrant compound. The seal may be made of any suitable material including polymeric materials and formed such that the seals on either the lid portion or the remaining portion insert into the other with a tight fit, or the seals press against each other under the force of a closure mechanism such as a clasp, screw, snap, or other mechanism, or the lid and remaining portion are mutually held against a single seal by a closure mechanism.

In certain embodiments configured to provide scent within the space of a room or vehicle, any of the modes and embodiments described above may be employed. The reservoir size may be increased for a longer duration of fragrance release as a convenience for the user. The attachment or clasping mechanism may be adapted to the features of the space such as trim pieces, ledges, shelves, air vents, brackets, stands, hooks, and other features that may or may not have been originally intended for attachment of a fragrance release device.

Exemplary embodiments include a device for releasing a fragrant compound, where the device comprises: a reservoir; a channeled member in fluid communication with the reservoir; and a fragrant compound disposed within the reservoir and in fluid communication with the channeled member, wherein the channeled member is configured to control a sustained release of the fragrant compound from the reservoir to the outside environment. In certain embodiments, the reservoir is detachable from the device. Particular embodiments further comprise a seal configured to retain the fragrant compound disposed within the reservoir.

In some embodiments, the device comprises a housing assembly; the reservoir is a separate component from the housing assembly; and the reservoir is configured to be coupled to the housing assembly such that the channeled member is in fluid communication with the reservoir when the reservoir is coupled to the housing assembly. In specific embodiments the housing assembly comprises a projection configured to puncture the seal when the reservoir is coupled to the housing assembly. In certain embodiments, the device comprises a housing assembly; the reservoir is a separate component from the housing assembly; the seal is detachable from the reservoir; and the reservoir is configured to be coupled to the housing assembly such that the channeled member is in fluid communication with the reservoir when the seal is detached from the reservoir and the reservoir is coupled to the housing assembly

In some embodiments, the device may be inverted to break the fluid communication between the reservoir and the channeled member to effectively slow the release of fragrant compound to the outside environment. In specific embodiments, the channeled member comprises nanochannels. In certain embodiments, the channeled member comprises microchannels. In particular embodiments, the channeled member is configured to release the fragrant compound over a time period of at least one hour, or over a time period of at least two hours, or over a time period of at least five hours, or over a time period of at least ten hours, or over a time period of at least twenty hours, or over a time period of at least two days, or over a time period of at least thirty days.

Some embodiments further comprise an inlet port in fluid communication with the reservoir, where the inlet port is configured to transfer the fragrant compound from a separate container to the reservoir. In certain embodiments, the inlet port is configured to couple to a nozzle configured to spray a mist. In particular embodiments, the inlet port is configured to couple to a funnel. In some embodiments, the channeled member is configured so that the fragrant compound is released from the reservoir in a gaseous state. In particular embodiments, the channeled member is configured so that the fragrant compound is released from the reservoir in a liquid state and subsequently evaporates from the surface of an evaporation diffuser. In specific embodiments, the channeled member is configured so that the fragrant compound is released from the reservoir in a liquid state and subsequently evaporates from the surface of the channeled member. Certain embodiments further comprise a coupling mechanism configured to couple the device to a personal article. In particular embodiments, the personal article is an article of clothing. In some embodiments, the personal article is a jewelry component. In specific embodiments, the jewelry component is a bracelet, a necklace, a brooch, a ring, or an earring.

Certain embodiments include a device for releasing a fragrant compound, where the device comprises: a reservoir; a channeled member in fluid communication with the reservoir, wherein the channeled member is configured to control a sustained release of a fragrant compound from the reservoir to the outside environment; and a coupling mechanism configured to couple the device to a personal article. In some embodiments, the reservoir is detachable from the device. Particular embodiments further comprise a seal configured to retain the fragrant compound disposed within the reservoir. In certain embodiments, the device comprises a housing assembly; the reservoir is a separate component from the housing assembly; and the reservoir is configured to be coupled to the housing assembly such that the channeled member is in fluid communication with the reservoir when the reservoir is coupled to the housing assembly.

In particular embodiments, the housing assembly comprises a projection configured to puncture the seal when the reservoir is coupled to the housing assembly. In certain embodiments, the device comprises a housing assembly; the reservoir is a separate component from the housing assembly; the seal is detachable from the reservoir; and the reservoir is configured to be coupled to the housing assembly such that the channeled member is in fluid communication with the reservoir when the seal is detached from the reservoir and the reservoir is coupled to the housing assembly. In some embodiments, the device may be inverted to break the fluid communication between the reservoir and the channeled member to effectively slow the release of fragrant compound to the outside environment.

In specific embodiments, the personal article is an article of clothing, or a hand-held electronic device, a cover for a hand-held electronic device, or a jewelry component. In certain embodiments the jewelry component is a bracelet, a necklace, a brooch, a ring, or an earring. In particular embodiments, the channeled member comprises nanochannels. In some embodiments, the channeled member comprises microchannels. In particular embodiments, the channeled member is configured to release the fragrant compound over a time period of at least one hour, or over a time period of at least two hours, or over a time period of at least five hours, or over a time period of at least ten hours, or over a time period of at least twenty hours, or over a time period of at least two days, or over a time period of at least thirty days

Some embodiments further comprise an inlet port in fluid communication with the reservoir, where the inlet port is configured to transfer the fragrant compound from a separate container to the reservoir. In particular embodiments, the inlet port is configured to couple to a nozzle configured to spray a mist. In some embodiments, the inlet port is configured to couple to a funnel. In specific embodiments, the channeled member is configured so that the fragrant compound is released from the reservoir in a liquid state and subsequently evaporates from the surface of the channeled member. In certain embodiments, the channeled member is configured so that the fragrant compound is released from the reservoir in a liquid state and subsequently evaporates from the surface of an evaporation diffuser. In particular embodiments, the channeled member is configured so that the fragrant compound is released from the reservoir in a gaseous state.

Certain embodiments include a method of providing a sustained release of a fragrant compound, the method comprising placing the fragrant compound in a reservoir of a device, where the device comprises a channeled member in fluid communication with the reservoir. Particular embodiments further comprise coupling the device to a personal article. In some embodiments, placing the fragrant compound in the reservoir of the device comprises: opening a cap of the device to provide access to the reservoir; and transferring the fragrant compound from a separate container to the reservoir. In specific embodiments, transferring the fragrant compound from the separate container to the reservoir comprises inserting a funnel into an inlet port in fluid communication with the reservoir. In certain embodiments, transferring the fragrant compound from the separate container to the reservoir comprises coupling a nozzle to an inlet port in fluid communication with the reservoir.

Particular embodiments include an apparatus for releasing a fragrant compound, where the apparatus comprises a personal article, and a device, where the device comprises: a reservoir; and a channeled member in fluid communication with the reservoir, where the channeled member is configured to control a sustained release of a fragrant compound from the reservoir to the outside environment; and the device is incorporated into the personal article. In some embodiments, the reservoir is detachable from the device. Particular embodiments further comprise a seal configured to retain the fragrant compound disposed within the reservoir. In certain embodiments, the device comprises a housing assembly; the reservoir is a separate component from the housing assembly; and the reservoir is configured to be coupled to the housing assembly such that the channeled member is in fluid communication with the reservoir when the reservoir is coupled to the housing assembly.

In particular embodiments, the housing assembly comprises a projection configured to puncture the seal when the reservoir is coupled to the housing assembly. In certain embodiments, the device comprises a housing assembly; the reservoir is a separate component from the housing assembly; the seal is detachable from the reservoir; and the reservoir is configured to be coupled to the housing assembly such that the channeled member is in fluid communication with the reservoir when the seal is detached from the reservoir and the reservoir is coupled to the housing assembly. In some embodiments, the device may be inverted to break the fluid communication between the reservoir and the channeled member to effectively slow the release of fragrant compound to the outside environment.

In specific embodiments, the personal article is an article of clothing, or a hand-held electronic device, a cover for a hand-held electronic device, or a jewelry component. In certain embodiments the jewelry component is a bracelet, a necklace, a brooch, a ring, or an earring. In particular embodiments, the channeled member comprises nanochannels. In some embodiments, the channeled member comprises microchannels. In particular embodiments, the channeled member is configured to release the fragrant compound over a time period of at least one hour, or over a time period of at least two hours, or over a time period of at least five hours, or over a time period of at least ten hours, or over a time period of at least twenty hours, or over a time period of at least two days, or over a time period of at least thirty days

Some embodiments further comprise an inlet port in fluid communication with the reservoir, where the inlet port is configured to transfer the fragrant compound from a separate container to the reservoir. In particular embodiments, the inlet port is configured to couple to a nozzle configured to spray a mist. In some embodiments, the inlet port is configured to couple to a funnel. In specific embodiments, the channeled member is configured so that the fragrant compound is released from the reservoir in a liquid state and subsequently evaporates from the surface of the channeled member. In certain embodiments, the channeled member is configured so that the fragrant compound is released from the reservoir in a liquid state and subsequently evaporates from the surface of an evaporation diffuser. In particular embodiments, the channeled member is configured so that the fragrant compound is released from the reservoir in a gaseous state.

In the following, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 10%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

As used herein, the term “sustained release” includes a release that lasts at least one or more hours and up to several days.

The term “personal article” as used herein includes articles worn by or kept near a person, including for example, clothing, jewelry, and handheld electronic devices.

The term “channeled member” as used herein includes structures comprising microchannels and/or nanochannels, including not limited to, any of the exemplary nanochannel devices disclosed in U.S. patent application Ser. No. 12/618,233 filed Nov. 13, 2009 (U.S. Patent Publication 20100152699) and entitled “Nanochanneled Device and Related Methods” and International Patent Application Number PCT/US10/30937 filed Apr. 13, 2010 and entitled “Nanochanneled Device and Method of Use”, both of which are incorporated herein by reference.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE FIGURES

FIG. 1 is a perspective view of an exemplary embodiment of a device for releasing a fragrant compound.

FIG. 2 is a perspective view of the embodiment of FIG. 1 in conjunction with other components.

FIG. 3 is a section view of an exemplary embodiment of a device for releasing a fragrant compound.

FIG. 4 is a section view of an exemplary embodiment of a device for releasing a fragrant compound in a first position.

FIG. 5 is a section view of the embodiment of FIG. 4 in a second position.

FIG. 6A is graph of release rate data of exemplary embodiments of a device for releasing a fragrant compound.

FIG. 6B is graph of release rate data of exemplary embodiments of a device for releasing a fragrant compound for a longer time period into a larger space.

FIG. 7 is a stylized representation of the scent experience of the top, middle, and base notes of a fragrant compound with conventional application and via embodiments of the present disclosure.

FIG. 8 is a perspective view of an exemplary embodiment of a device for releasing a fragrant compound.

FIG. 9 is a perspective view of an exemplary embodiment of a device for releasing a fragrant compound.

FIG. 10 is a perspective view of an exemplary embodiment of a device for releasing a fragrant compound.

FIG. 11 is a perspective view of an exemplary embodiment of a device for releasing a fragrant compound.

FIG. 12 is a perspective view of an exemplary embodiment of a device for releasing a fragrant compound during use.

FIG. 13 is a perspective view of an exemplary embodiment of a device for releasing a fragrant compound during use.

FIG. 13 is a perspective view of an exemplary embodiment of a device for releasing a fragrant compound during use.

FIG. 14 is a perspective view of an exemplary embodiment of a device for releasing a fragrant compound during use.

FIG. 15 is a perspective view of an exemplary embodiment of a device for releasing a fragrant compound during use.

### DETAILED DESCRIPTION OF EXEMPLARY EMBODIMENTS

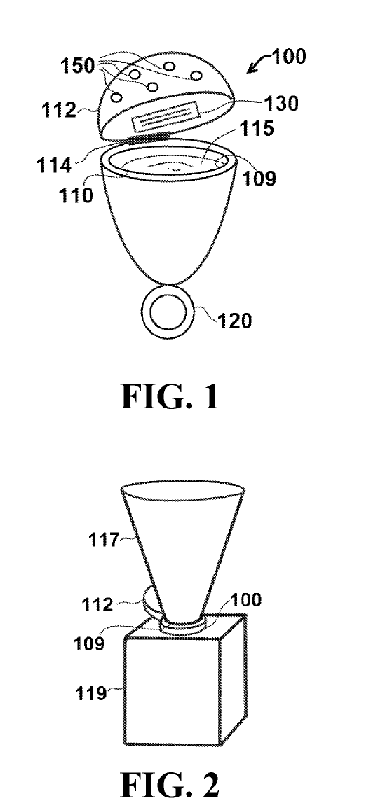

Referring now to FIGS. 1-2, an exemplary embodiment of a device 100 comprises a reservoir 110, a channeled member 130 and a plurality of exit ports 150. In the embodiment shown, a fragrant compound 115 is disposed within reservoir 110 and channeled member 130 is configured to control a sustained release of fragrant compound 115 from reservoir 110 to the outside environment. In certain embodiments, device 100 may comprise a cap 112 that can be opened or closed via a hinge 114 to access an inlet port 109 to reservoir 110. It is understood that the cap and hinge arrangement shown in FIG. 1 is just one example of a configuration to provide access to the reservoir, and other embodiments may have different configurations, including for example, a cap that snaps onto the device and can be completely separated to provide access to the reservoir.

The embodiment shown also comprises a coupling mechanism 120 configured to couple the device to a personal article, including for example an article of clothing or a jewelry component. In certain embodiments, coupling mechanism 120 may be a ring, clasp, hook or other suitable mechanism used for coupling device 100 to a personal article. In specific embodiments, the personal article may be a blouse, shirt, pants, undergarment, or other article of clothing, or bracelet, necklace, brooch, watch, ring, earring, cell phone, eye glasses frame, or other jewelry or worn or carried accessory or an enclosure case that protects handheld devices like cell-phones, tablets, or laptops.

During use, fragrant compound 115 (e.g. a perfume or cologne) can be placed in reservoir 110 (through various methods described more fully below) and cap 112 closed. Coupling mechanism 120 can then be coupled to a personal article worn by the user. In order for fragrant compound 115 to be released to the outside environment, it first passes through channeled member 130 and then exits ports 150. In exemplary embodiments, exit ports 150 are sized sufficiently large so that they do not restrict or control the release of fragrant compound 115 from reservoir 110. Channeled member 130, however, can comprise nanochannels and/or microchannels configured to control a sustained release of fragrant compound 115 to the outside environment. In certain embodiments, channeled member can comprise microchannels having at least one dimension within that is less than 5 μm to control the release of fragrant compound 115. In certain other embodiments, channeled member can comprise nanochannels having at least one dimension within that is less than 500 nm to control the release of fragrant compound 115.

The sustained release of fragrant compound 115 (e.g. over multiple hours or even days) can provide a consistent fragrant or aromatic level over a period of time for a user and others in the vicinity of the user. Exemplary embodiments of device 100 are capable of providing a sustained release of fragrant compound 115 such that it is noticeable to a user over a period of time of one hour, two hours, five hours, ten hours, twenty hours or even two or more days. Typical applications of perfumes, colognes or other fragrant compounds involve applying the compounds directly to the skin or clothing of a user. Such use can provide for an initially high aromatic level that is greater than desired (and can be unpleasant for the user or others nearby), but which quickly dissipates to a level that is less than desired. The dissipation of fragrance level over time can require a user to frequently reapply the fragrant compound in order to maintain a desirable fragrance level and fragrance mix of top, middle, and base notes over an extended period of time.

Exemplary embodiments of device 100 utilize channeled member 130 to restrict the diffusion of molecules of fragrant compound 115 (as fragrant compound 115 evaporates) to the outside environment. This can reduce or eliminate the likelihood that the initial aromatic levels of fragrant compound 115 will be greater than desired. In addition, reservoir 110 can provide the capacity for a sufficient volume of fragrant compound 115 to maintain a sustained release of the compound at desirable levels over multiple hours or days.

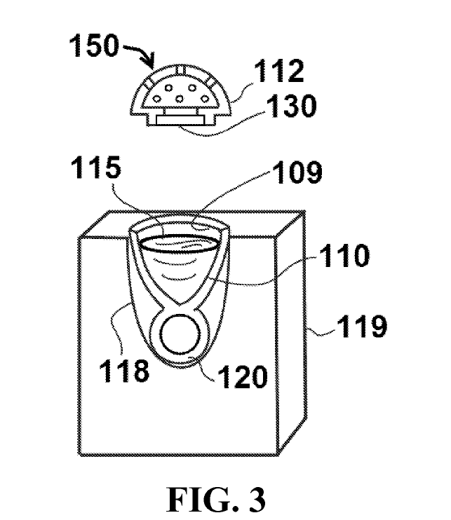

Referring specifically now to FIG. 3, in certain embodiments reservoir 110 of device 100 can be filled via a funnel 117 and a loading stand 119. Specifically, device 100 can be placed in a receptacle 118 in stand 119 and cap 112 opened so that funnel 117 can be inserted into inlet port 109 to provide access to reservoir 110 (not visible in FIG. 2 due to the placement of funnel 117). A fragrant compound can then be placed into reservoir 110 via funnel 117. In certain embodiments, the fragrant compound may be poured through funnel 117 into reservoir 110, while in other embodiments the fragrant compound may be sprayed into funnel 117 such that the droplets of fragrant compound collect on funnel 117 and run into reservoir 110, and in such embodiments the funnel 117 may have a coating, e.g., a hydrophobic coating, that facilitates sprayed droplets that land on the funnel 117 running into the reservoir 110. In still other embodiments, reservoir 110 may be filled with a fragrant compound in other manners, including for example a connector to a standard aromatic spritzing nozzle that allows the nozzle to direct the fragrant compound to the reservoir. After fragrant compound 115 has been placed in reservoir 110, cap 112 can be closed to cover reservoir 110. Device 100 can then be removed from loading stand 119 and coupled to a personal article via coupling mechanism 120. Fragrant compound 115 can then be released from device 100 by diffusing and evaporating through channeled member 130 and exit ports 150. As previously described, exemplary embodiments can comprise a channeled member 130 with nanochannels or microchannels that are dimensioned to control the release of fragrant compound 115 in order to provide a sustained release of fragrant compound 115.

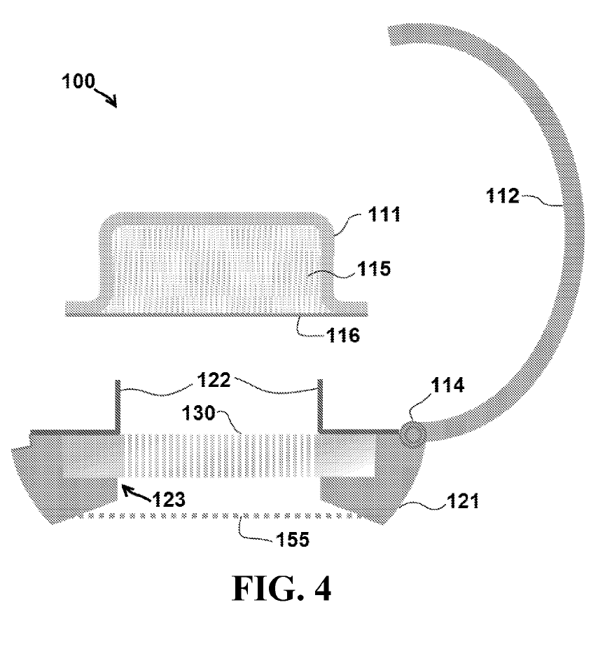

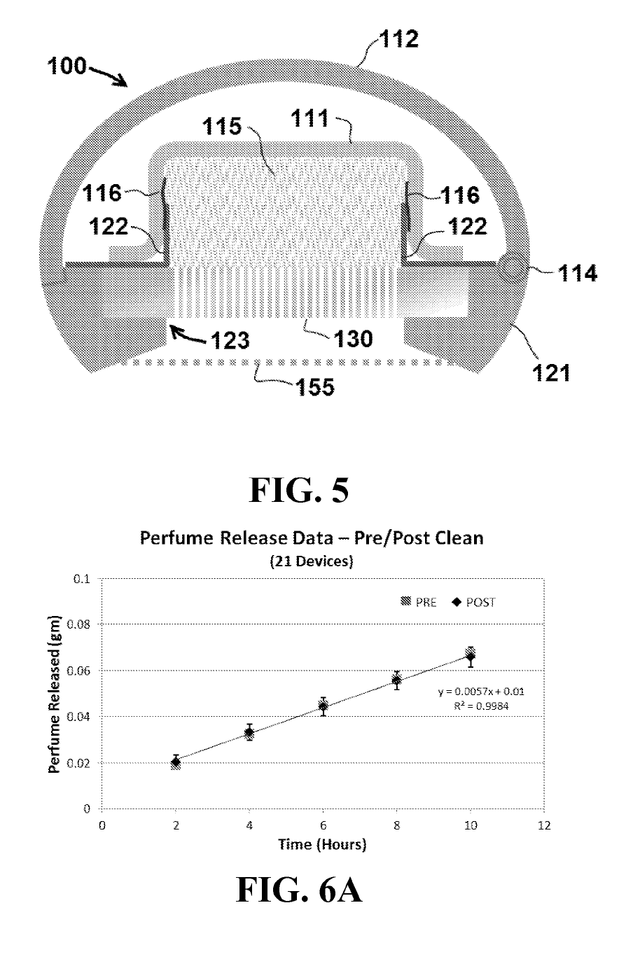

Referring now to FIGS. 4-5, in certain embodiments, device 100 may comprise a reservoir 111 that is detachable from device 100 and capable being refilled and/or replaced. In the embodiment shown, channeled member 130 coupled to a housing 121 with cap 112 that can be opened via hinge 114. In particular embodiments, channeled member 130 may be coupled to housing 121 by gluing, overmolding, gasketing, or other coupling methods to form a housing assembly 123. In the illustrated embodiment shown, device 100 comprises reservoir 111 that is a detachable and separate component from housing assembly 123. In this embodiment, reservoir 111 also comprises a seal 116 configured to retain fragrant compound 115 within reservoir 111 until reservoir 111 is coupled to channeled member 130 and housing assembly 123.

In certain embodiments, seal 116 may be one of or a combination of metal foil, polymeric sheet, or other film impervious to fragrant compound 115. In particular embodiments, seal 116 may be detached or removed from reservoir 111 prior to coupling reservoir 111 to housing assembly 123. In other embodiments, housing assembly 123 may comprise one or more projections 122 configured to puncture seal 116 when reservoir 111 is coupled to housing assembly 123. In either case, reservoir 111 is coupled to housing assembly 123 such that fragrant compound 115 exits reservoir 111 via channeled member 130 (rather than the interface between reservoir 111 and housing assembly 123).

In an embodiment in which seal 116 is punctured, projection 122 and residual material of seal 116 can form a fluid tight seal system when pressed into fully mated position shown in FIG. 5, such that fragrant compound 115 is restricted from escaping at the interface of reservoir 111 and housing assembly 123, but allowing reservoir 111 to be subsequently removed for replacement. In the embodiment shown in FIGS. 4-5, housing assembly 123 also comprises a screen 155 between channeled member 130 and the outside environment. Similar to exit ports 150 of previously-described embodiments, screen 155 includes openings sufficiently large so that they do not restrict or control the release of fragrant compound 115 from reservoir 111. During use, the release of fragrant compound 115 to the outside environment is controlled in a manner equivalent to that described in previous embodiments.

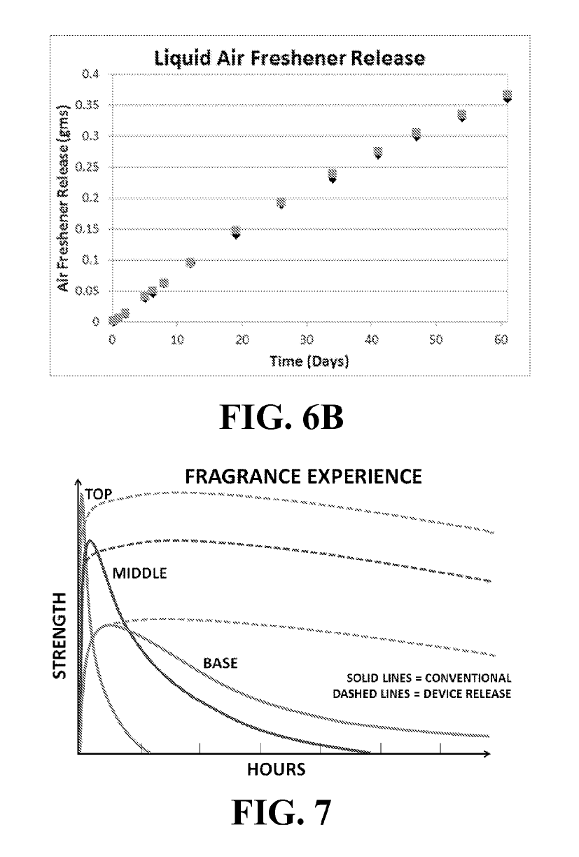

Referring now to FIG. 6A, the release rate (measured in grams) is shown over a ten hour time frame for exemplary embodiments of devices according to the present disclosure. Data was collected from twenty one different devices by measuring the weight of the device with the fragrant compound at two hour intervals. In the graph shown in FIG. 6A, the gray squares represent data obtained from an initial loading of a fragrant compound into device. After the initial data set was obtained, the devices were cleaned with ethanol and re-loaded with the fragrant compound. The release rate was again measured over a ten hour period at two-hour intervals. The post-cleaning release data is represented by black diamonds in the graph. As shown in FIG. 6A, the release rate post-cleaning was virtually identical to the release rate pre-cleaning. This data indicates that exemplary embodiments of the device may be cleaned and re-used without substantially affecting the release rate of the fragrant compound contained within the device. Precise channel members in the nano/micro regime resulted in a repetitive controlled fragrance release behavior with very little variations, as evidenced by the small error bars in the graph.

Referring now to FIG. 6B, the release rate (measured in grams) is shown over a sixty day time frame for two nominally identical exemplary embodiments of devices according to the present disclosure. In this embodiment the fragrant compound released was a liquid air freshener. The gray squares represent data from the first embodiment and the black diamonds represents data from the second embodiment. As shown in the figure, the data for the first embodiment was consistent with the data from the second embodiment.

The data illustrated in FIGS. 6A and FIG. 6B also indicates that devices according to the present disclosure provide a consistent, sustained release rate of a fragrant compound over extended periods of time (e.g. ten hours or 60 days). Such a sustained release is in contrast to conventional application techniques for fragrant compounds, which provide initially strong fragrance levels that quickly dissipate over time. FIG. 7 provides a stylized representation of the scent experience of the top, middle, and base notes of a fragrant compound under conventional application (solid lines) and via embodiments of the present disclosure (dashed lines).

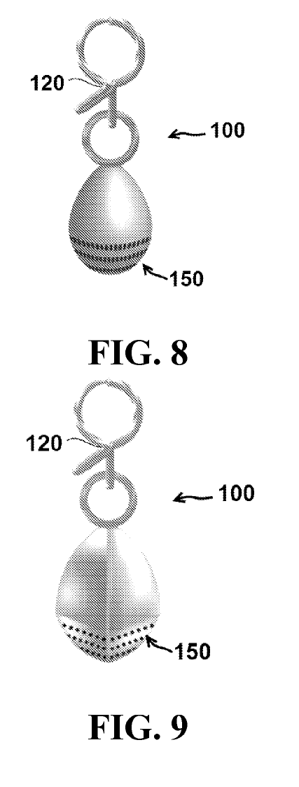

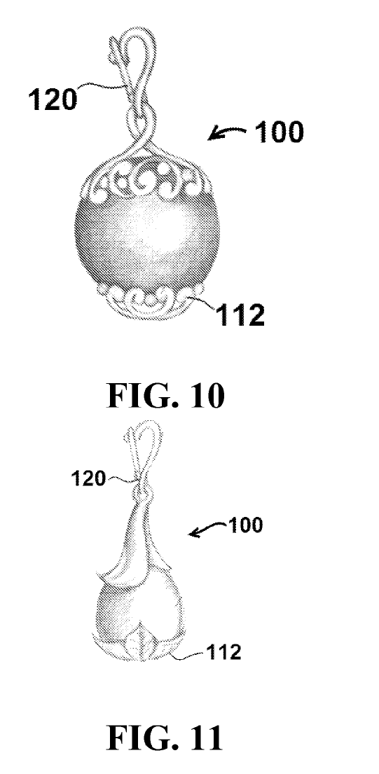

In particular embodiments, device 100 can be configured as an ornamental device that can be worn as jewelry. In certain embodiments device 100 may include an outer layer or coating of yellow or white metal, including gold or other precious metal. Referring now to FIG. 8, one exemplary embodiment of device 100 may be formed in a general teardrop shape with exit ports 150 arranged in a decorative pattern. As shown in FIG. 9, another embodiment of device 100 may comprise a squared teardrop shape with exit ports 150 also arranged in a decorative pattern. The embodiments shown in FIGS. 10 and 11 illustrate examples of other ornamental designs, including decorative designs for cap 112. It is understood that not all features of the embodiments (for example, exit ports 150) are visible in FIGS. 10-11, but are included in configurations. The embodiments shown in FIGS. 8-11 also comprise a coupling mechanism 120 configured to allow device 100 to be coupled to a personal article.

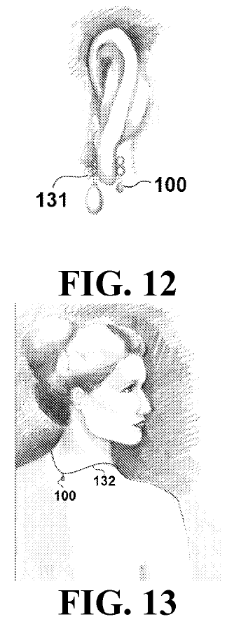

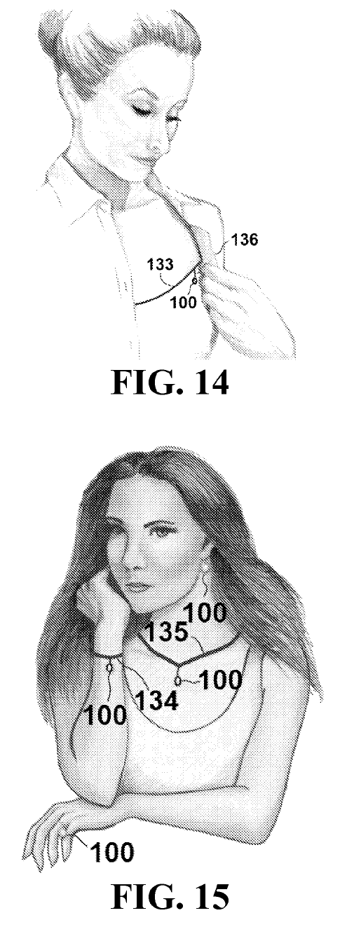

Referring now to FIGS. 12-15, device 100 is shown being utilized in various manners and coupled to different types of personal articles. For example, as shown in FIG. 12, device 100 is coupled to the back of an earring 131. In FIG. 13, device 100 is coupled to the back of a necklace 132, while in FIG. 14 device 100 is coupled to a shirt 133 (or other clothing garment) that is obscured by a blouse 136. In the embodiments shown in FIGS. 12-14, device 100 is not prominently displayed and therefore the appearance of device 100 is a secondary consideration.

Referring now to FIG. 15, device 100 can be worn as an ornamental jewelry component and coupled to the front of a necklace 135 or to a bracelet 134 to be prominently displayed by the user. In the embodiment shown in FIG. 15, device 100 can provide an aesthetically pleasing function as ornamental jewelry, as well as provide improved aromatic levels by dispensing a fragrant compound.

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein: U.S. Patent Publication 20100152699

1. A device for releasing a fragrant compound, the device comprising:
a reservoir;
a channeled member in fluid communication with the reservoir; and
a fragrant compound disposed within the reservoir and in fluid communication with the channeled member, wherein the channeled member is configured to control a sustained release of the fragrant compound from the reservoir to the outside environment.

2. The device of claim 1 wherein the reservoir is detachable from the device.

3. The device of claim 1 further comprising a seal configured to retain the fragrant compound disposed within the reservoir.

4. The device of claim 3 wherein:
the device comprises a housing assembly;
the reservoir is a separate component from the housing assembly; and
the reservoir is configured to be coupled to the housing assembly such that the channeled member is in fluid communication with the reservoir when the reservoir is coupled to the housing assembly.

5. The device of claim 4 wherein the housing assembly comprises a projection configured to puncture the seal when the reservoir is coupled to the housing assembly.

6. The device of claim 3 wherein:
the device comprises a housing assembly;
the reservoir is a separate component from the housing assembly;
the seal is detachable from the reservoir; and
the reservoir is configured to be coupled to the housing assembly such that the channeled member is in fluid communication with the reservoir when the seal is detached from the reservoir and the reservoir is coupled to the housing assembly.

7. The device of claim 1 wherein the device may be inverted to break the fluid communication between the reservoir and the channeled member to effectively slow the release of fragrant compound to the outside environment.

8. The device of claim 1 wherein the channeled member comprises nanochannels.

9. The device of claim 1 wherein the channeled member comprises microchannels.

10. The device of claim 1 wherein the channeled member is configured to release the fragrant compound over a time period of at least one hour.

11. The device of claim 1 wherein the channeled member is configured to release the fragrant compound over a time period of at least two hours.

12. The device of claim 1 wherein the channeled member is configured to release the fragrant compound over a time period of at least five hours.

13. The device of claim 1 wherein the channeled member is configured to release the fragrant compound over a time period of at least ten hours.

14. The device of claim 1 wherein the channeled member is configured to release the fragrant compound over a time period of at least twenty hours.

15. The device of claim 1 wherein the channeled member is configured to release the fragrant compound over a time period of at least two days.

16. The device of claim 1 wherein the channeled member is configured to release the fragrant compound over a time period of at least thirty days.

17. The device of claim 1 further comprising an inlet port in fluid communication with the reservoir, wherein the inlet port is configured to transfer the fragrant compound from a separate container to the reservoir.

18. The device of claim 17 wherein the inlet port is configured to couple to a nozzle configured to spray a mist.

19. The device of claim 17 wherein the inlet port is configured to couple to a funnel.

20. The device of claim 1 wherein the channeled member is configured so that the fragrant compound is released from the reservoir in a gaseous state.

21. The device of claim 1 wherein the channeled member is configured so that the fragrant compound is released from the reservoir in a liquid state and subsequently evaporates from the surface of an evaporation diffuser.

22. The device of claim 1 wherein the channeled member is configured so that the fragrant compound is released from the reservoir in a liquid state and subsequently evaporates from the surface of the channeled member.

23. The device of claim 1 further comprising a coupling mechanism configured to couple the device to a personal article.

24. The device of claim 23 wherein the personal article is an article of clothing.

25. The device of claim 23 wherein the personal article is a jewelry component.

26. The device of claim 25 wherein the jewelry component is a bracelet.

27. The device of claim 25 wherein the jewelry component is a necklace.

28. The device of claim 25 wherein the jewelry component is a brooch.

29. The device of claim 25 wherein the jewelry component is a ring.

30. The device of claim 25 wherein the jewelry component is an earring.

31. A device for releasing a fragrant compound, the device comprising:
a reservoir;
a channeled member in fluid communication with the reservoir, wherein the channeled member is configured to control a sustained release of a fragrant compound from the reservoir to the outside environment; and
a coupling mechanism configured to couple the device to a personal article.

32. The device of claim 31 wherein the reservoir is detachable from the device.

33. The device of claim 31 further comprising a seal configured to retain the fragrant compound disposed within the reservoir.

34. The device of claim 33 wherein:
the device comprises a housing assembly;
the reservoir is a separate component from the housing assembly; and
the reservoir is configured to be coupled to the housing assembly such that the channeled member is in fluid communication with the reservoir when the reservoir is coupled to the housing assembly.

35. The device of claim 34 wherein the housing assembly comprises a projection configured to puncture the seal when the reservoir is coupled to the housing assembly.

36. The device of claim 33 wherein:
the device comprises a housing assembly;
the reservoir is a separate component from the housing assembly;
the seal is detachable from the reservoir; and
the reservoir is configured to be coupled to the housing assembly such that the channeled member is in fluid communication with the reservoir when the seal is detached from the reservoir and the reservoir is coupled to the housing assembly.

37. The device of claim 31 wherein the device may be inverted to break the fluid communication between the reservoir and the channeled member to effectively slow the release of fragrant compound to the outside environment.

38. The device of claim 31 wherein the personal article is an article of clothing.

39. The device of claim 31 wherein the personal article is a hand-held electronic device.

40. The device of claim 31 wherein the personal article is a cover for a hand-held electronic device.

41. The device of claim 31 wherein the personal article is a jewelry component.

42. The device of claim 41 wherein the jewelry component is a bracelet.

43. The device of claim 41 wherein the jewelry component is a necklace.

44. The device of claim 41 wherein the jewelry component is a brooch.

45. The device of claim 41 wherein the jewelry component is a ring.

46. The device of claim 41 wherein the jewelry component is an earring.

47. The device of claim 31 wherein the channeled member comprises nanochannels.

48. The device of claim 31 wherein the channeled member comprises microchannels.

49. The device of claim 31 wherein the channeled member is configured to release the fragrant compound over a time period of at least one hour.

50. The device of claim 31 wherein the channeled member is configured to release the fragrant compound over a time period of at least two hours.

51. The device of claim 31 wherein the channeled member is configured to release the fragrant compound over a time period of at least five hours.

52. The device of claim 31 wherein the channeled member is configured to release the fragrant compound over a time period of at least ten hours.

53. The device of claim 31 wherein the channeled member is configured to release the fragrant compound over a time period of at least twenty hours.

54. The device of claim 31 wherein the channeled member is configured to release the fragrant compound over a time period of at least two days.

55. The device of claim 31 wherein the channeled member is configured to release the fragrant compound over a time period of at least thirty days.

56. The device of claim 31 further comprising an inlet port in fluid communication with the reservoir, wherein the inlet port is configured to transfer the fragrant compound from a separate container to the reservoir.

57. The device of claim 56 wherein the inlet port is configured to couple to a nozzle configured to spray a mist.

58. The device of claim 56 wherein the inlet port is configured to couple to a funnel.

59. The device of claim 31 wherein the channeled member is configured so that the fragrant compound is released from the reservoir in a liquid state and subsequently evaporates from the surface of the channeled member.

60. The device of claim 31 wherein the channeled member is configured so that the fragrant compound is released from the reservoir in a liquid state and subsequently evaporates from the surface of an evaporation diffuser.

61. The device of claim 31 wherein the channeled member is configured so that the fragrant compound is released from the reservoir in a gaseous state.

62. A method of providing a sustained release of a fragrant compound, the method comprising placing the fragrant compound in a reservoir of a device, wherein the device comprises a channeled member in fluid communication with the reservoir.

63. The method of claim 62 further comprising coupling the device to a personal article.

64. The method of claim 62 wherein placing the fragrant compound in the reservoir of the device comprises:
opening a cap of the device to provide access to the reservoir; and
transferring the fragrant compound from a separate container to the reservoir.

65. The method of claim 64 wherein transferring the fragrant compound from the separate container to the reservoir comprises inserting a funnel into an inlet port in fluid communication with the reservoir.

66. The method of claim 64 wherein transferring the fragrant compound from the separate container to the reservoir comprises coupling a nozzle to an inlet port in fluid communication with the reservoir.

67. An apparatus for releasing a fragrant compound, the apparatus comprising:
a personal article; and
a device comprising:
a reservoir; and
a channeled member in fluid communication with the reservoir, wherein:
the channeled member is configured to control a sustained release of a fragrant compound from the reservoir to the outside environment; and
the device is incorporated into the personal article.

68. The apparatus of claim 67 wherein the reservoir is detachable from the device.

69. The apparatus of claim 67 further comprising a seal configured to retain the fragrant compound disposed within the reservoir.

70. The apparatus of claim 69 wherein:
the device comprises a housing assembly;
the reservoir is a separate component from the housing assembly; and
the reservoir is configured to be coupled to the housing assembly such that the channeled member is in fluid communication with the reservoir when the reservoir is coupled to the housing assembly.

71. The apparatus of claim 70 wherein the housing assembly comprises a projection configured to puncture the seal when the reservoir is coupled to the housing assembly.

72. The apparatus of claim 69 wherein:
the device comprises a housing assembly;
the reservoir is a separate component from the housing assembly;
the seal is detachable from the reservoir; and
the reservoir is configured to be coupled to the housing assembly such that the channeled member is in fluid communication with the reservoir when the seal is detached from the reservoir and the reservoir is coupled to the housing assembly.

73. The apparatus of claim 67 wherein the device may be inverted to break the fluid communication between the reservoir and the channeled member to effectively slow the release of fragrant compound to the outside environment.

74. The apparatus of claim 67 wherein the personal article is an article of clothing.

75. The apparatus of claim 67 wherein the personal article is a hand-held electronic device.

76. The apparatus of claim 67 wherein the personal article is a cover for a hand-held electronic device.

77. The apparatus of claim 67 wherein the personal article is a jewelry component.

78. The apparatus of claim 77 wherein the jewelry component is a bracelet.

79. The apparatus of claim 77 wherein the jewelry component is a necklace.

80. The apparatus of claim 77 wherein the jewelry component is a brooch.

81. The apparatus of claim 77 wherein the jewelry component is a ring.

82. The apparatus of claim 77 wherein the jewelry component is an earring.

83. The apparatus of claim 67 wherein the channeled member comprises nanochannels.

84. The apparatus of claim 67 wherein the channeled member comprises microchannels.

85. The apparatus of claim 67 wherein the channeled member is configured to release the fragrant compound over a time period of at least one hour.

86. The apparatus of claim 67 wherein the channeled member is configured to release the fragrant compound over a time period of at least two hours.

87. The apparatus of claim 67 wherein the channeled member is configured to release the fragrant compound over a time period of at least five hours.

88. The apparatus of claim 67 wherein the channeled member is configured to release the fragrant compound over a time period of at least ten hours.

89. The apparatus of claim 67 wherein the channeled member is configured to release the fragrant compound over a time period of at least twenty hours.

90. The apparatus of claim 67 wherein the channeled member is configured to release the fragrant compound over a time period of at least two days.

91. The apparatus of claim 67 wherein the channeled member is configured to release the fragrant compound over a time period of at least thirty days.

92. The apparatus of claim 67 further comprising an inlet port in fluid communication with the reservoir, wherein the inlet port is configured to transfer the fragrant compound from a separate container to the reservoir.

93. The apparatus of claim 92 wherein the inlet port is configured to couple to a nozzle configured to spray a mist.

94. The apparatus of claim 92 wherein the inlet port is configured to couple to a funnel.

95. The apparatus of claim 67 wherein the channeled member is configured so that the fragrant compound is released from the reservoir in a liquid state and subsequently evaporates from the surface of the channeled member.

96. The apparatus of claim 67 wherein the channeled member is configured so that the fragrant compound is released from the reservoir in a liquid state and subsequently evaporates from the surface of an evaporation diffuser.
